(19) United States
(12) Patent Application Publication
(10) Pub. No.: US 2024/0000598 A1
(43) Pub. Date: Jan. 4, 2024

(54) Adjustable Back Supporter

(71) Applicant: Linwood F. Hamilton, Charleston, WV (US)

(72) Inventor: Linwood F. Hamilton, Charleston, WV (US)

(21) Appl. No.: 18/118,268

(22) Filed: Mar. 7, 2023

Related U.S. Application Data
(60) Provisional application No. 63/317,093, filed on Mar. 7, 2022.

(51) Int. Cl. A61F 5/02

(52) U.S. Cl. CPC A61F 5/02

(57) ABSTRACT

A back supporter is designed to be worn by anyone who needs support in the bending position is described and may include selectable range limiters the user can select to restrict the motion of the hinge. The back supporter is to be worn on the front of the body from mid-chest to mid-thigh. The back supporter is hinged at the waist with supports that allow the wearer to bend to a certain degree and stop. In some embodiments the back supporter may include hinges that provide for the unlimited adjustment of the bending position for the back supporter. The back support takes pressure off of the lower back of the user and reduces fatigue and lower back injuries.

### CROSS-REFERENCE TO RELATED APPLICATIONS

This application claims the benefit of U.S. Provisional Patent Application No. 63/317,093, filed Mar. 7, 2022, specifically incorporated herein in its entirety.

### FIELD OF THE INVENTION

The present invention is directed to a back supporter to support the back of a user while the user in the bending position for extended periods of time.

### SUMMARY OF THE INVENTION

Embodiments of the present invention include a back supporter for a user comprising an upper portion adapted to cover the front portion of user extending from the hips towards the shoulder of the user, a lower portion adapted to cover the front portion of user extending from the hips towards the knees of the user, and a hinge connecting the upper portion and lower portion, wherein the hinge is adjustable and limits the movement of the upper portion relative to the lower portion when the user bend forward. In some embodiments the hinge may include a lever for adjusting the position of the hinge in a predetermined position. Still further, the hinge may include a friction insert position between an inner arm pivot head and an outer arm pivot head to help hold the hinge in a fixed position by the user.

Embodiments of the invention may include a hinge for an orthopedic support comprising an inner arm having an inner arm pivot head, wherein the inner arm is adapted to be connected to a first portion of the support; an outer arm having an outer arm pivot head, wherein the outer arm is adapted to be connected to a second portion of the support, and wherein the inner arm pivot head and the outer arm pivot head are connected through a pivot point; and at least one adjustment pin adapted to extend through the outer arm pivot head and into an adjustment pin slot defined in the inner arm pivot head when engaged by the user. In certain embodiments, the hinge may include a fixed range pin extending through the outer arm pivot head and into a fixed range slot defined in the inner arm.

### BRIEF DESCRIPTION OF THE DRAWINGS

The importance of the features and merits of the present invention will be better understood by reference to the attached drawings. It is to be understood, however, that the drawings are designed for the purpose of illustration only and not as a definition of the limits of the present invention.

FIG. 1 is a diagrammatic view of a back supporter in accordance with an embodiment of the invention shown on a user.

FIG. 2 is a diagrammatic side view of a back supporter in accordance with an embodiment of the invention.

FIG. 3 is a diagrammatic view of a hinge in accordance with an embodiment of the invention.

FIG. 4A is a diagrammatic rear view of a hinge in accordance with an embodiment of the invention.

FIG. 4B is a diagrammatic side view of a hinge in accordance with an embodiment of the invention.

FIG. 4C is a diagrammatic front view of a hinge in accordance with an embodiment of the invention.

FIG. 4D is a diagrammatic end view of a hinge in accordance with an embodiment the invention.

FIG. 4E is a diagrammatic end view of a hinge from the opposite end illustrated in FIG. 4D, in accordance with an embodiment the invention.

FIG. 5 is a diagrammatic top view of a hinge in accordance with another embodiment of the invention.

FIG. 6 is a diagrammatic side view of a hinge in accordance with another embodiment of the invention.

### DETAILED DESCRIPTION OF EMBODIMENTS OF THE INVENTION

Some preferred embodiments of the present invention are described in this section in detail sufficient for one skilled in the art to practice the present invention without undue experimentation.

It is to be understood, however, that the fact that a limited number of preferred embodiments are described in this section does not in any way limit the scope of the present invention as set forth in the claims.

It is to be understood that whenever a range of values is described herein, i.e. whether in this section or any other part of this patent document, that the range includes the end points and every point therebetween as if each and every such point had been expressly described. Unless otherwise stated, the words “about” and “substantially” as used herein are to be construed as meaning the normal measuring and/or fabrication limitations related to the value or condition which the word “about” or “substantially” modifies. Unless expressly stated otherwise, the term “embodiment” is used herein to mean an embodiment of the present invention.

The present invention is directed to a to a back supporter to support the back of a user while the user in a bending position for extended periods of time.

The present invention is directed to an adjustable back supporter designed to be worn by anyone who needs support in the bending position for prolonged periods of time. It is to be worn on the front of the body from mid-chest to mid-thigh. It is hinged at the waist with supports that allow the wearer to bend to a certain degree and stop. This will take pressure off of the lower back. Saving the back will reduce fatigue and lower back injuries. Advantageously, the hinge is adapted to be locked in a primary supported position while on the user.

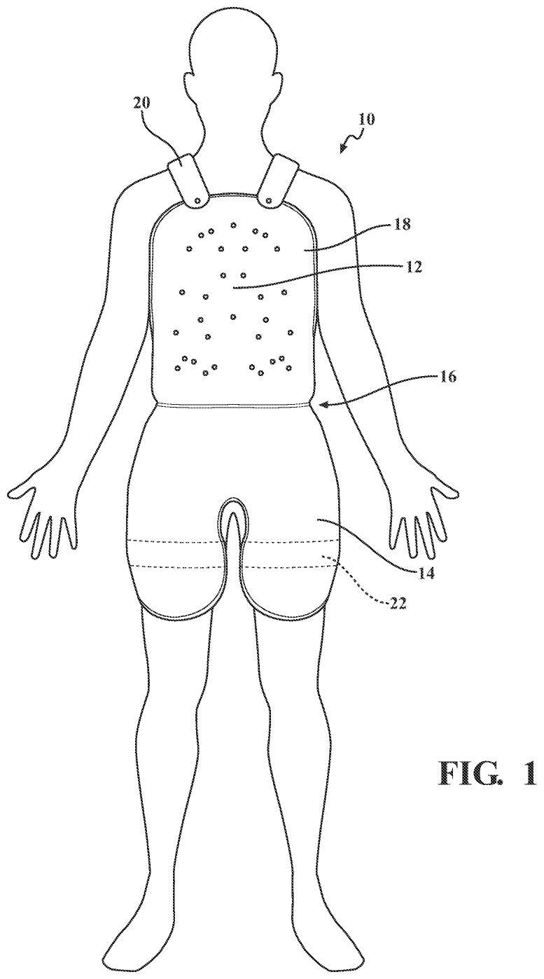

With reference now to FIG. 1, there is shown a back supporter 10 in accordance with an embodiment of the invention positioned on a user. The back supporter 10 includes an upper portion 12 and a lower portion 14 connected to the upper portion by a hinge 16. The upper portion 12 is adapted to cover a portion of the front torso of a user. In certain embodiments the upper portion 12 begins from about the hips of the user and extends towards the shoulders of the user. The lower portion 14 is adapted to cover a portion of the front torso of a user beginning from about the hips of the user and extending towards the knees of the user. As used herein, the “hips” of the user is that region of the user that allows for and provide the ability of the user to bend the upper torso forward. The upper portion 12 and lower portion 14 may be contoured to the approximated shape of the user taking into consideration general anatomical shapes and curves of the user. The upper portion 12 and lower portion 14 may be customized to a particular user or alternatively may contain certain general anatomical shapes and curves. The upper portion 12 and the lower portion 14 may also contain holes or perforations 18 to allow ventilation for the user while the back supporter 10 is being worn by the user. Further, cushioning material such as a foam may be used on the upper and lower portions to provide a move comfortable fit.

The upper portion 12 and the lower portion 14 may be made from any material suitable to hold or support the weight of the user. Suitable materials may include but are not limited to metals, plastics, and composite materials. Plastics such as polyethylene or polypropylene may be used to form the upper and/or lower portions 12 and 14 respectively. Preferably the materials are lightweight materials to minimize the fatigue of a user for wearing the back supporter 10. In certain embodiments the upper portion 12 and the lower portion 14 are made from a moldable plastic, such as those used for injection molding. In other embodiments, the upper portion and lower portion may be made from a composite material such as a fiber reinforced composite such as carbon fiber or glass fibers embedded in a polymer matrix. The upper portion 12 and the lower portion 14 may be made from the same or different materials.

The upper portion 12 and the lower portion 14 are connected by a hinge 16. The hinge 16 allows restricted movement of the upper portion 12 relative to the lower portion 14. The hinge 16 allows the upper portion 12 to move and form an angle with the lower portion 14 when the user is bending forward. In certain embodiments the hinge 16 limits the movement of the upper portion 12 such that when the user in bending forward, the upper portion 12 will move and form an angle with the lower portion 14 to a certain point at which the hinge 16 will no longer allow the upper portion 12 to continue to move in the direction of the user's bend. In this way, the user may “rest” on the upper portion 12 while in a bending position. In certain embodiments, the hinge 16 may be adjustable such the degree of forward movement of the upper portion 12 may be adjustable based on the needs of the user. In some embodiments, the hinge may be made from metal. In other embodiments the hinge 16 may be made from plastic.

In further embodiments, the hinge 16 includes a locking mechanism such as a button or lever to lock the hinge 16 in a predetermined position desired by the user. Preferably the locking mechanism is easily accessible to the user while wearing the back supporter 10 such that the locking mechanism may be engaged and disengaged at anytime while being worn by the user.

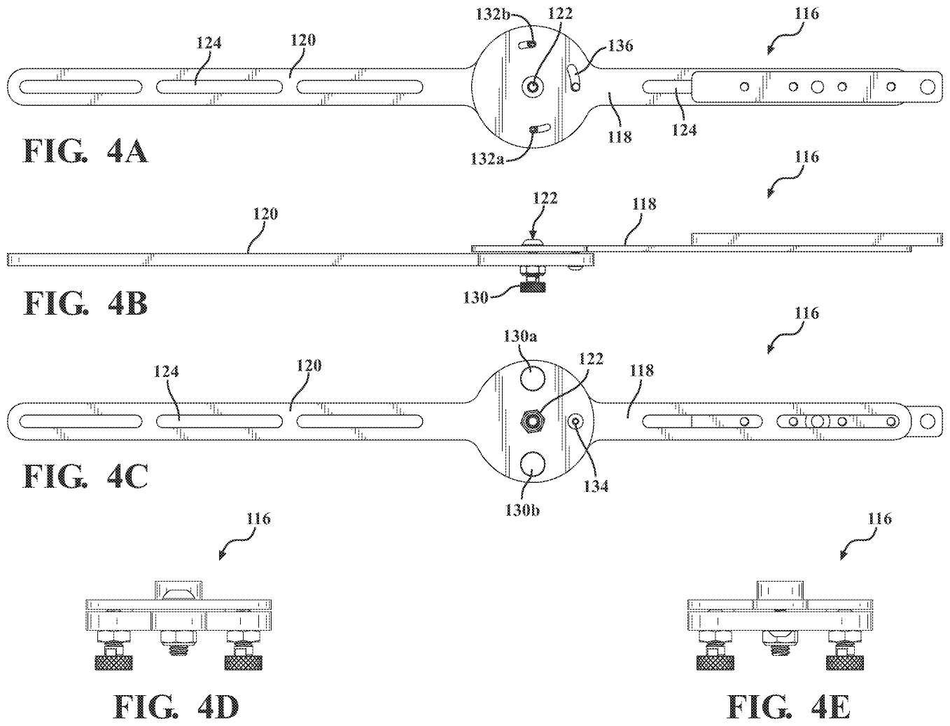

With reference now to FIG. 3 and FIG. 4A-4E, there is shown a hinge 116 in accordance with an embodiment of the invention. The hinge is a pivot style hinge, sometimes known as a knife hinge or scissor hinge. The hinge 116 includes an inner arm 118 and an outer arm 120 connected at a pivot point 122. The inner arm and outer arm define a plurality of attachment holes or slots 124 that allow connection to an exoskeletal suit such as the back supporter 10 having an upper portion 12 and a lower portion 14 connected by a hinge. Regular hardware such as a nut and bolt may be used to connect the arms of the hinge to the upper portion and lower portion of the suit.

The arms of the hinge are provided with a pivot head. The outer arm 120 includes an outer arm pivot head 126. The inner arm 118 includes an inner arm pivot head 128. The outer arm pivot head 126 and the inner arm pivot head 128 are connected through pivot point 122. Pivot point 122 may include a nut and bolt extending through the outer arm pivot head 126 and inner arm pivot head 128 through the pivot point 122.

The hinge 116 includes at least one, preferably 2, and in some embodiments a plurality of adjustment pins. With reference to FIG. 4A-4C, the hinge 116 is provided with adjustment pins 130a and 130b. The adjustment pins 130a and 130b extend through the outer arm pivot head 126 and into adjustment pin slots 132a and 132b defined in the inner arm pivot head 128 to restrict the travel of the respective adjustment pin when the hinge arms of the hinge are moved about the pivot point. The adjustment pins 130 A and 1:30 B may include a head that is accessible by the user or operator, and a pin extending from the head. When the head is depressed by the user and locked in place, the pin extending from the head extends through the outer arm pivot head 126 and into the respective adjustment pin slot defined in the inner arm pivot head 128. The length of the adjustment slot limits the movement of the outer arm 120 with respect to the inner arm 118 when the respective adjustment pin is engaged or depressed. The length of the adjustment pin slot may vary from one adjustment slot to the other allowing different ranges of motion depending on what combination of adjustment pins or engaged or otherwise depressed.

As will be appreciated, the invention may include more than one adjustment pin and respective adjustment pin slots to allow for various range adjustment limitations for the hinge. With reference to the embodiment shown in FIG. 4A-4B, two adjustment pins 130a and 130b and respective adjustment pin slots 132a and 132b are provided for the hinge 116 . The length of adjustment pin slot 132a is longer than the length of adjustment pin slot 132b. This provides the user with two range limitations for movement of the inner arm 118 with respect to the outer arm 120. When adjustment pin 130b is depressed or otherwise engaged, the pin extends into adjustment pin slot 132b. The movement of the inner arm 118 relative to the outer arm 120 is thus limited by the length of the adjustment slot 132b. Similarly, if adjustment pin 130a is depressed, the movement of the inner arm 118 relative to the outer arm 120 is limited by the length of adjustment pin slot 132a. Since the length of adjustment pin slot 132a is longer then the length of adjustment pin slot 132b, the hinge will have a greater range of motion when adjustment pin 130a is engaged. In this way, the user is able to select how much range of motion is desired through the selection of which adjustment pins are engaged. This has the advantage of providing a selectable but limited range of motion for the hinge. This can be desirable for the back support are 10 as described above, but also for any orthopedic device in which some range of motion is desired yet needs to be limited within a certain range.

In some embodiments one or more of the adjustment pins maybe fixed and not engageable by the user. With reference to FIG. 4A-4C, a fixed range pin 134 is provided. The fixed range pin 134 extends from the outer arm pivot head 126 into a fixed range pin slot 136 defined in the inner arm pivot head 128. Since the fixed range pin 134 is designed to provide the maximum allowable range of movement for the hinge 116. The length of the fixed range slot 136 is longer than any of the other adjustment slots provided. In this way if no adjustment pins 130a or 130b are engaged by the user, the range of movement of the hinge will be limited by the fixed range pin 134 and the fixed range slot 136. Because the inner arm 118 and the outer arm 120 are rotated relative to one another about the pivot point 122, the adjustment slots and fixed range slot are in the form of an arc about the pivot point to allow for ease of rotation about the pivot point.

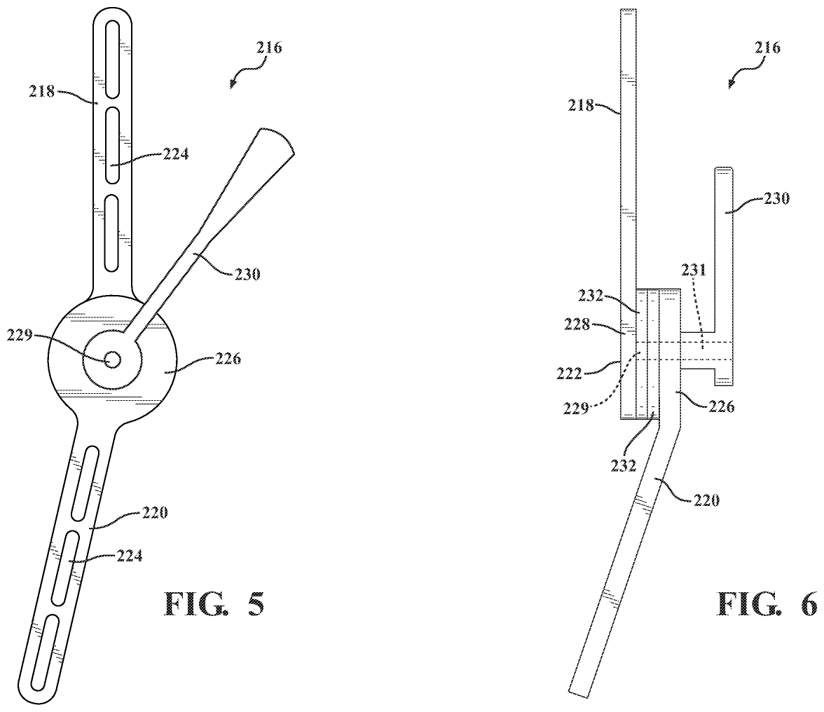

With reference to FIG. 5 and FIG. 6, there is illustrated a hinge 216 in accordance with another embodiment of the invention. The hinge 216 includes an inner arm 218 and an outer arm 220 connected at a pivot point 222. The inner arm and outer arm define a plurality of attachment holes or slots 224 that allow connection to an exoskeletal suit such as the back supporter 10 having an upper portion 12 and a lower portion 14 connected by a hinge. The arms of the hinge are provided with a pivot head. The outer arm 220 includes an outer arm pivot head 226. The inner arm 218 includes an inner arm pivot head 228. The outer arm pivot head 226 and the inner arm pivot head 228 are connected through pivot point 222. The inner arm pivot head 228 may include a shaft 229 extending through the pivot point 222 and through the outer arm pivot head 226. A distal portion of the shaft extending through outer arm pivot head 226 is threaded. A lever 230 is threadably connected to the distal portion 231 of the shaft extending through the outer arm pivot head 226. In certain embodiments, the lever 230 has a series of female threads matching the threads on the distal portion 231 of the shaft and effectively acts as a nut on the end of a threaded shaft. Moving the lever 230 in one direction tightens the pivot point 222 thus restricting the movement of the hinge 216. Moving lever 230 in the opposite direction loosens the pivot point 222 and allows the hinge to move. In this way, the lever may be used to adjust the position of the outer arm 220 and inner arm 218 to any suitable angle for the user.

In some embodiments one of the outer arm 220 and inner arm 218 may be bent at an angle relative to the other. Bending either of the outer arm 220 or inner arm 218 may in some embodiments provide a better fit around the hip area of a user when the user is wearing the back supporter 10.

In certain embodiments the hinge 216 may also include at least one and preferably a pair of friction inserts 232 positioned between the outer arm pivot head 226 and the inner arm pivot head 228. The friction inserts 232 provide increase friction for the pivot point 222 to reduce movement of the outer arm 220 and inner arm 218 once the lever 230 has tightened the pivot point 222. The friction inserts 232 may be made from any abrasive material that increases the friction between the outer arm pivot head 226 and the inner arm pivot head 228. The friction inserts may be sand paper, polymer non-woven abrasives, ceramic abrasives, metallic abrasives, textured or roughened metallic surfaces (including surfaces outer arm pivot head 226 and the inner arm pivot head 228).

The hinge 116 or 216 may be made any durable material able to withstand these stress and forces applied to the hinge. Metals including high strength aluminum or aluminum alloys, titanium or titanium alloys, steel alloys and the like may be utilized. Additionally, the hinge may be made from high strength polymers, polymer composites, or fiber reinforced polymers.

In additional embodiments the hinge 16 may optionally include a counterweight mechanism (not shown) such as a spring or other tensioning material. As the user is bending forward, the counterweight mechanism provides a force towards the user. In this way, the user encounters resistance being provided by the counterweight mechanism while bending forward. In certain embodiments, the force is sufficient to support the weight the user is exerting on the upper portion 12 when bending at an angle of about 30 to about 90 degrees from vertical. Similarly, hinges with tensioning springs or materials are known to those skilled in the art and may be used with the present invention provided they meet the criteria of being able support the users weight and be able to be locked in a predetermined position as discussed above.

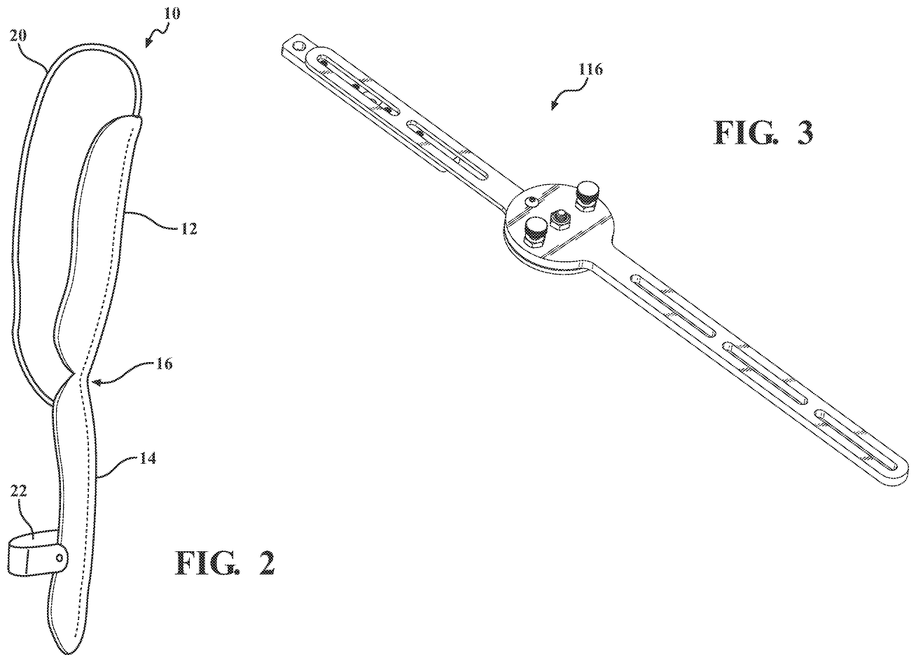

With reference to FIG. 2 and continued reference to FIG. 1, to assist in maintaining the position of the upper portion 12 and lower portion 14 on the user, upper straps 20 and lower straps 22 may be used. In certain embodiments upper straps 20 are adapted to hold the upper portion 12 in place on the user. The upper straps 20 preferably include a pair of straps that connect to a top edge 24 of the upper portion 12 and connect to a top edge 26 of the lower portion 14. The upper straps 20 are adapted to go over the shoulders of the user. Preferably the upper straps 20 are adjustable to allow for correct positioning of the back supporter 10 on the user.

Lower straps 22 are connected to the lower portion 14 and adapted to go around one or more legs of the user. Lower straps 22 may include a pair of lower straps as shown in FIG. 1 that secure the lower portion 14 to each leg of the user, or alternatively, only one strap may be used to secure one or both legs of the user. Preferably the lower straps 22 are adjustable to allow for correct positioning of the back supporter 10 on the user.

The upper straps 20 and lower straps 22 may be made from any material known to those skilled in the art for producing straps or adjustable straps. The straps may be made from materials, including but not limited to, leather, natural or synthetic fabrics. To provide adjustability of the upper straps 20 and lower straps 22, a traditional belt and buckle system may be used or more preferably a hook and loop fastening system may be used.

While only a few embodiments of the present invention have been shown and described, it will be obvious to those skilled in the art that many changes and modifications may be made thereunto without departing from the spirit and scope of the invention as described in the claims. All United States patents and patent applications, all foreign patents and patent applications, and all other documents identified herein are incorporated herein by reference as if set forth in full herein to the full extent permitted under the law.

What is claimed is:

1. A back supporter for a user comprising:
An upper portion adapted to cover the front portion of user extending from the hips towards the shoulder of the user, a lower portion adapted to cover the front portion of user extending from the hips towards the knees of the user, and a hinge connecting the upper portion and lower portion, wherein the hinge is adjustable and limits the movement of the upper portion relative to the lower portion when the user bends forward, and wherein the hinge comprises a locking mechanism for locking the position of the hinge in a predetermined position.

2. The back supporter of claim 1, wherein the hinge further comprises a counterweight mechanism.

3. The back support of claim 1, wherein the hinge further comprises a lever for adjusting the position of the hinge in a predetermined position.

4. The back support of claim 3, wherein the hinge further comprises a friction insert position between an inner arm pivot head and an outer arm pivot head.

5. A hinge for an orthopedic support comprising:
an inner arm having an inner arm pivot head, wherein the inner arm is adapted to be connected to a first portion of the support;
an outer arm having an outer arm pivot head, wherein the outer arm is adapted to be connected to a second portion of the support, and wherein the inner arm pivot head and the outer arm pivot head are connected through a pivot point; and
at least one adjustment pin adapted to extend through the outer arm pivot head and into an adjustment pin slot defined in the inner arm pivot head when engaged by the user.

6. The hinge of claim 5, further comprising:
a fixed range pin extending through the outer arm pivot head and into a fixed range slot defined in the inner arm.
